(19) United States
(12) Patent Application Publication
(10) Pub. No.: US 2003/0198571 A1
(43) Pub. Date: Oct. 23, 2003

(54) Ozone sterilization process an apparatus for denaturing prions

(76) Inventor: Edward Lynch, Belfast (GB)

Correspondence Address: WALTER A. HACKLER, 2372 S.E. BRISTOL, SUITE B, NEWPORT BEACH, CA, 92660-0755, US

(21) Appl. No.: 10/350,753

(22) Filed: Jan. 24, 2003

Related U.S. Application Data
(60) Provisional application No. 60/355,332, filed on Feb. 6, 2002.

(51) Int. Cl. A61L 9/00; A62B 7/08; B01J 19/08; A61L 2/00

(52) U.S. Cl. 422/28; 422/30; 422/32; 422/34; 422/121; 422/186.07; 422/292; 422/305

(57) ABSTRACT

Apparatus is provided for the sterilization of Prion infected surgical tools and the method for such sterilization includes disposing Prion infected surgical tools in the chamber, evacuating the chamber and introducing ozone into the chamber for a period of time sufficient to oxidize Prion amino acids. During this exposure the tools are agitated in order to insure all surface areas are exposed to the ozone. Following sterilization the ozone and sterilized tools are removed from the chamber.

The present invention is generally related to a method and apparatus for the sterilization of surgical tools. More particularly the present invention relates to a method and apparatus for the sterilization of surgical tools infected with Prions.

Aseptic conditions are critically important in most surgical activities. Sterilization of materials and tools are of utmost necessity in biological, medical and dental applications.

Sterilization methods in use today primarily include heating, ultraviolet radiation, nuclear radiation and chemical disinfection. Autoclave heating has been commonly used because of its availability and the simplicity of operation.

Unfortunately, high temperatures and steam concomitant with steam autoclaving places further demands on the materials which can be successfully autoclaved. In fact autoclave sterilization of materials in tools which include plastics have a very limited application.

Techniques involving chemical sterilization often are limited by toxic residue and require elaborate safety procedures.

Ultraviolet radiation and other radiation techniques, such as nuclear radiation, require involved apparatus and often custom installations.

The hereinabove described techniques, however, do not successfully remove or denature Prions. Prions are amino acids that are unusually resistant to conventional disinfectant procedures.

For example an electrode used intracerebrally on a Creutzfeldt-Jakob Disease (CJD) patient is known to transmit the disease to subsequent patients despite attempted disinfection.

The present invention provides for a method and apparatus for sterilization of Prion infected surgical tools.

### SUMMARY OF THE INVENTION

A method in accordance with the present invention for the sterilization of Prion infected surgical tools includes the steps of disposing Prion infected surgical tools into a chamber and thereafter evacuating the chamber.

Ozone is introduced into the chamber for period of time sufficient to oxidize Prion amino acids. Thereafter the ozone is removed from the chamber and the sterilized surgical tools are also removed from the chamber.

Apparatus in accordance with the present invention for the sterilization of objects generally includes a sealed chamber sized for receiving surgical instruments and means for agitating the surgical instruments within the chamber.

An ozone source is connected to the chamber for introducing ozone into the evacuated chamber during agitation of the surgical instruments. The agitation insures that all surfaces of the surgical tools are exposed to the ozone for oxidation of the Prion amino acids.

A system is then provided for removing ozone from the chamber after sterilization of the instruments and inactivating the ozone.

### BRIEF DESCRIPTION OF THE DRAWINGS

The advantages and features of the present invention will be better understood by the following description when considered in conjunction with the accompanying drawings, in which:

FIG. 1 illustrates a block diagram of apparatus for carrying out the method of the present invention which includes the sterilization of Prion infected surgical tools, the apparatus generally including a sealable chamber, a vacuum system for evacuating air from the chamber, an ozone source connected to the chamber for introducing ozone into an evacuated chamber, a system for removing ozone gas from the chamber after sterilization and means for agitating a surgical instruments within the chamber to insure sterilization of all surface areas.

### DETAILED DESCRIPTION

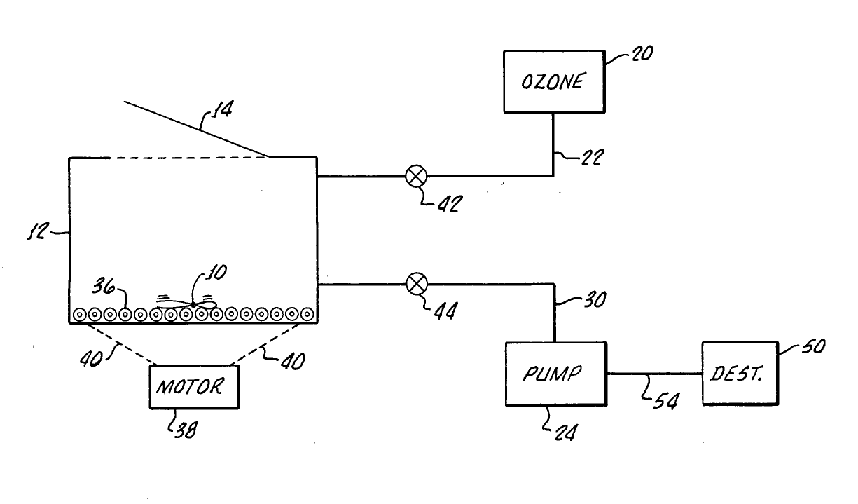

With reference to FIG. 1 there shown apparatus 10 in accordance with the present invention for the sterilization of objects and more particularly for the sterilization of Prion infected surgical tools by oxidation of Prion amino acids.

The apparatus 10 generally includes a chamber 12 which is sealable by way of the door 14 following the placement of a surgical tool 16 within the chamber 12. An ozone source, or generator, 20 is provided which may be of any suitable type which is interconnected with the chamber 12 by a line 22. As used herein, the term “ozone” is intended to embrace any suitable oxidizing gas such as pure ozone, ionized air and other ozone gaseous mixtures. A pump 28 and line 30 provide a vacuum system for evacuating air from the chamber 12 after sealing the chamber 12 by closure of the door 14.

A set of rollers 36 interconnected with a motor as indicated by dashed lines 40 provide a means for agitating the surgical instrument, or tool, 16 within the chamber 12 during exposure to ozone generated by the ozone source 20 and supplied through the line 22. Control valves 42 44 may be provided for controlling, along with the pump 28 and ozone source 20, ozone and air flow in to and out of the chamber 12.

While represented as rollers 36 in FIG. 1 it must be appreciated that any suitable means for agitating the tool 16 within the chamber 12 may be utilized and is encompassed within the scope of the present invention. A canister 50 including manganese or other reducing agent in combination with the pump 28 line 30 and valve 44 provide a system for removing ozone form the chamber 12 after sterilization of the instruments 16. The reducing agent effectively inactivates the ozone and accordingly the apparatus 10 is a closed system with regard to ozone, that is, no ozone escapes from the apparatus 10 during or after the sterilization procedure.

A method in accordance with the present invention for the sterilization of Prion infected surgical tools utilizing the equipment 10 includes disposing the Prion infected surgical tool 16 within the chamber 12, sealing the chamber by way of the door 14 and evacuating air from the chamber 12 via the line 30 and valve 44 by means of the pump 28.

Following evacuation ozone is introduced into the chamber 12 by way of the line 22 and valve 42 from the ozone source 20. The ozone remains in the chamber for a period of time sufficient to oxidize Prion amino acids. The period of ozone exposure is typically between about 2 minutes and about 10 minutes.

After sterilization the ozone is removed from the chamber by the pump 28 through the line 30 and valve 44 and introduced by a line 54 into the canister 50. As hereinabove noted canister 50 includes a reducing agent for reducing ozone and combination with the pump 28 as hereinabove described effectively removes all of the ozone from the chamber 12 without released to the atmosphere.

As noted the surgical tool 16 is agitated by the rollers 36 in order to insure that all surface areas are exposed to the ozone within the chamber 12.

Alternatively the tool 16 maybe suspended or otherwise disposed within the chamber 12 in a manner that all surface areas are exposed to ozone.

Although there has been hereinabove described apparatus method for the sterilization of surgical tools in accordance with the present invention for the purpose of illustrating the manner in which the invention may be used to advantage, it will be appreciated that the invention is not limited thereto. Accordingly, any and all modifications, variations or equivalent arrangements which may occur to those skilled in the art, should be considered to be within the scope of the invention as defined in the appended claims.

What is claimed is:

1. A method for sterilization of prior infected surgical tools, said method comprising the steps of:
disposing prion infected surgical tools into a chamber;
evaluating the chamber;
introducing ozone into the chamber for a period of time sufficient to oxidize prion amino acids;
removing the ozone from the chamber; and
removing sterilized surgical tools from the chamber.

2. The method according to claim 1 wherein the tools are agitated within the chamber during the period of time ozone is present in the chamber.

3. Apparatus for the sterilization of objects, said apparatus comprising:
a sealable chamber sized for receiving surgical instruments;
means for agitating the surgical instruments within the chamber;
a vacuum system for evacuating air from the chamber;
an ozone source, connected to the chamber, for introducing ozone into an evacuated chamber during agitation of the surgical instruments; and
a system for removing ozone from the chamber after sterilization of the instruments and inactivating ozone.
